(19) United States
(12) Patent Application Publication
(10) Pub. No.: US 2016/0018378 A1
(43) Pub. Date: Jan. 21, 2016

(54) SYSTEMS AND METHODS FOR BOOSTING COAL QUALITY MEASUREMENT STATEMENT OF RELATED CASES

(71) Applicant: Siemens Aktiengesellschaft, München (DE)

(72) Inventors: Chao Yuan, Plainsboro, NJ (US); Amit Chakraborty, East Windsor, NJ (US); Holger Hackstein, Dietzenbach (DE); Ping Zhang, Elkins Park, PA (US); Liang Lan, Philadelphia, PA (US)

(21) Appl. No.: 14/772,088

(22) PCT Filed: Feb. 13, 2014
(86) PCT No.: PCT/US2014/016177

§ 371 Date: Sep. 2, 2015

Related U.S. Application Data
(60) Provisional application No. 61/773,915, filed on Mar. 7, 2013.
(60) Provisional application No. 61/773,932, filed on Mar. 7, 2013.
(60) Provisional application No. 61/774,805, filed on Mar. 8, 2013.

(51) Int. Cl. G01N 33/22; G01N 21/359; G01N 21/3563

(52) U.S. Cl. CPC G01N 33/222; G01N 21/3563; G01N 21/359

(57) ABSTRACT

Properties of coal are determined from samples processed by a near-infrared spectroscopy (NIR) device that generates wavelengths dependent spectra. Target values of the properties are associated with the NIR spectra by a kernel based regression model generated from training data based on an anisotropic kernel function that is extended by defining the kernel parameters as a smooth function over the wavelengths associated with a spectrum. Like the anisotropic case each wavelength related dimension has its own kernel parameter. Adjacent dimensions are restricted to have similar kernel parameters. Measured spectra with a limited number of features are reconstructed by applying a regression model based on training data of spectra having an extended number of features. Training data are pruned based on a regression model by removing outliers.

### STATEMENT OF RELATED CASES

The present application claims priority to and the benefit of U.S. Provisional Patent Application Ser. No. 61/773,915 filed on Mar. 7, 2013, of U.S. Provisional Patent Application Ser. No. 61/773,932 filed on Mar. 7, 2013 and of U.S. Provisional Patent Application Ser. No. 61/774,805 filed on Mar. 8, 2013, which are all three incorporated herein by reference in their entirety.

### TECHNICAL FIELD

The present invention is related to systems and methods for improving measuring the quality of coal. More in particular it relates to methods and systems for improving regression based methods in determining a coal quality with Near-Infrared Spectroscopy.

### BACKGROUND

Knowing the content of the coal such as the concentration of H2O or heatan is of great importance to the energy industry because more efficient control and optimization strategies can be applied to the boiler accordingly. Directly measuring these quantities is often prohibitive due to the high cost.

In contrast, using coal spectrum produced by Near-Infrared spectroscopy (NIR) is less expensive and more practical. However, a spectrum doesn't directly provide the target values of the desired physical quantities. A following procedure is often employed. In a first stage being a training stage, a regression function is learned from the spectrum to the ground truth target value. In a second stage being a material testing (or implementation) stage, only the spectrum of an unknown coal is given and the learned regression function is applied to predict the target value.

Learning this regression function is challenging for several reasons. A Near-Infrared Spectroscopy spectrum usually consists of readings from thousands of wavelengths and often only a limited number of ground truth target values is available, for instance due to the cost of measuring these values. Also determining a complete and extensive spectrum, beyond for a limited number of training samples is not economical. Furthermore, noise and other influences may create outliers in measurement results that skew the accuracy of the regression models.

Present regression models applied in determining coal quality do not adequately address these issues.

Accordingly, novel and improved regressions methods and systems to improve the measurement of coal quality with Near-Infrared Spectroscopy are required.

The following references describe or illustrate aspects current methodologies in regression based modeling and are incorporated herein by reference:
[1] S. An, W. Liu, and S. Venkatesh. Fast cross-validation algorithms for least squares support vector machine and kernel ridge regression. Pattern Recognition, 40(8):2154-2162, 2007; [2] C. E. Rasmussen and C. K. I. Williams. Gaussian Processes for Machine Learning. MIT Press, 2006; [3] Roman Rosipal and Leonard J. Trejo. Kernel partial least squares regression in reproducing kernel hilbert space. Journal of Machine Learning Research, 2:97-123, 2001; [4] B. Scholkopf, R. Herbrich, and A. J. Smola. A generalized representer theorem. In Proceedings of the 14th Annual Conference on Computational Learning Theory, pages 416-426, 2001; [5] S. Wold, H. Rube, H. Wold, and W. J. Dunn III. The collinearity problem in linear regression, the partial least squares (pis) approach to generalized inverse. SIAM Journal of Scientific and Statistical Computations, 5:735-743, 1984; and [3] T. Chen, and J. Ren. Bagging for Gaussian process regression. Neurocomputing, 72(7-9):1605-1610, 2009.

### SUMMARY

In accordance with various aspects of the present invention systems and methods are provided for boosting coal quality measurement.

In accordance with a further aspect of the present invention a method is provided for determining a property of a material from data generated by a near-infrared spectroscopy device, comprising: obtaining wavelength based training data related to the material, a processor using the wavelength based training data to learn an anisotropic Gaussian kernel function with a wavelength based kernel parameter that is defined by a smooth function over the wavelength determined by at least one parameter and the processor applying the anisotropic Gaussian kernel function to wavelength based test data of one or more samples of the material generated by the near-infrared spectroscopy device to determine the property.

In accordance with yet a further aspect of the present invention a method is provided, wherein the smooth function is a smooth Gaussian function and the at least one parameter is a decay parameter.

In accordance with yet a further aspect of the present invention a method is provided, wherein the material is coal.

In accordance with yet a further aspect of the present invention a method is provided, wherein the property is heatan.

In accordance with yet a further aspect of the present invention a method is provided, wherein the wavelength based kernel parameter that is defined by a smooth Gaussian function over the wavelength, is expressed as γ(d)=γ₀ exp(−β(l(d)−l₀)²), wherein d is an index value related to the wavelength, γ(d) is the wavelength based parameter, γ₀ is a maximum value of the wavelength based parameter, β is the decay parameter, l(d) is the wavelength at index value d, and l₀ is a wavelength value for which the wavelength based parameter reaches the maximum value.

In accordance with yet a further aspect of the present invention a method is provided, further comprising the processor learning a kernel ridge regression for an isotropic kernel from the training data, the processor determining a regularization factor and γ₀, the processor applying an initialization value for β and determining l₀ and the processor determining an operational value for β.

In accordance with yet a further aspect of the present invention a method is provided, further comprising the processor applying the kernel ridge regression to the wavelength based training data to determine a first plurality of target values, the processor determining a standard deviation from the first plurality of target values, the processor identifying a reduced plurality of sets of training data by removing at least one set of training data from the wavelength based training data based on the standard deviation and the processor applying the kernel ridge regression to the reduced plurality of sets of training data to determine a second plurality of target values.

In accordance with another aspect of the present invention a method is provided to reconstruct a feature in test data related to a material obtained with a near-infrared spectroscopy device, comprising: storing on a memory near-infrared spectroscopy training data from the material including data of a first and a second set of features which do not overlap, creating with a processor a predictive feature model to predict features appearing in the second set of features in the training data from the first set of features in the training data by using the first and second set of features in the training data, obtaining with the near infra-red spectroscopy device test data from the material including test data related to the first set of features and predicting a second set of features related to the test data of the material by applying the predictive feature model.

In accordance with yet another aspect of the present invention a method is provided, further comprising combining the first set of features and the predicted second set of features related to the test data to create a predictive model for a property of the material.

In accordance with yet another aspect of the present invention a method is provided, wherein each first set of features relates to a first range of wavelengths in NIR spectroscopy and each second set of features relates to a second range of wavelengths in NIR spectroscopy.

In accordance with yet another aspect of the present invention a method is provided, wherein the first range of wavelengths includes wavelengths shorter than 2300 nm and the second range of wavelengths includes wavelengths greater than 2300 nm

In accordance with yet another aspect of the present invention a method is provided, wherein the predictive feature model is based on a multivariate statistical method.

In accordance with yet another aspect of the present invention a method is provided, wherein the multivariate statistical method is a kernel ridge regression method.

In accordance with yet another aspect of the present invention a method is provided, wherein the material is coal and the property is a calorific value.

In accordance with a further aspect of the present invention a method is provided for determining a property of a material with data generated by a spectroscopy device, comprising a processor receiving a first plurality of sets of training data generated by the spectroscopy device, the processor generating a regression model from the first plurality of sets of training data to determine a first plurality of target values, which is representative of the property of the material, the processor determining a standard deviation from the first plurality of target values, the processor identifying a second plurality of sets of training data by removing at least one set of training data from the first plurality of sets of training data based on the standard deviation and the processor generating a regression model from the second plurality of sets of training data to determine a second plurality of target values.

In accordance with yet a further aspect of the present invention a method is provided, further comprising the processor generating a regression model from a remaining plurality of sets of training data to determine a remaining plurality of target values, the processor determining a new standard deviation from the remaining plurality of target values and the processor determining if any of the sets of training data of the remaining plurality of sets of training data should be removed based on the new standard deviation.

In accordance with yet a further aspect of the present invention a method is provided, wherein none of the sets of training data is removed from the remaining plurality of sets of training data and the regression model based on the remaining plurality of sets of training data is applied by the processor to determine a target value from a set of test data generated by the spectroscopy device.

In accordance with yet a further aspect of the present invention a method is provided, wherein the material is coal and the spectroscopy device is a near-infrared spectroscopy device.

In accordance with yet a further aspect of the present invention a method is provided, wherein the removing of at least one set of training data from the first plurality of sets of training data is based on a 3σ range.

In accordance with yet a further aspect of the present invention a method is provided, wherein the property is a calorific value of coal.

### DRAWINGS

FIG. 1 illustrates a spectrum in accordance with an aspect of the present invention.

FIG. 2 illustrates various steps in accordance with one or more aspects of the present invention.

FIG. 3 illustrates a smooth function in accordance with an aspect of the present invention.

FIG. 4 illustrates various steps in accordance with one or more aspects of the present invention.

FIG. 5 illustrates a plurality of spectra in accordance with various aspects of the present invention.

FIG. 6 illustrates a reconstructed spectrum in accordance with an aspect of the present invention.

FIG. 7 illustrates a plurality of spectra in accordance with various aspects of the present invention.

FIG. 8 illustrates outliers in accordance with various aspects of the present invention.

FIG. 9 illustrates various steps in accordance with one or more aspects of the present invention.

FIGS. 10A-10F illustrate pruning of training data in accordance with one or more aspects of the present invention.

FIG. 11 illustrates a processor based system in accordance with one or more aspects of the present invention.

### DESCRIPTION

Methods and processor based systems are provided herein in accordance with various aspects of the present invention to improve the determination of coal quality from samples with Near-Infrared Spectroscopy (NIR) devices and methods.

A coal quality measure such as water content or heatan content (=calorific heat value of the coal) is a property that is derived from an NIR spectrum with a regression model that is usually trained on ground truth data.

In accordance with various aspects of the present invention new regression methods and systems are provided.

Learning a regression function is challenging due to the following reasons. First, the measured spectrum usually consists of readings from thousands of wavelengths and often only a very limited number of ground truth target values is available (due to the cost of measuring these values). Therefore, this problem suffers from the curse of dimensionality. Second, the relation between the spectrum and the target value is observed to be nonlinear. So many standard linear algorithms such as partial least square (PLS) do not perform very well.

Nonlinear kernel regression algorithms such as kernel ridge regression (KRR) as described in “[1] S. An, W. Liu, and S. Venkatesh. Fast cross-validation algorithms for least squares support vector machine and kernel ridge regression. Pattern Recognition, 40(8):2154-2162, 2007” or Gaussian process (GP) as described in “[2] C. E. Rasmussen and C. K. I. Williams Gaussian Processes for Machine Learning. MIT Press, 2006” have produced the state-of-the-art results on this task.

One of the most widely used kernel functions is the Gaussian kernel, which is constructed either using an isotropic kernel parameter (one for all input dimensions) or using anisotropic kernel parameters (one for each of the input dimensions). The isotropic case is often over-simplified and ignores the differences among different wavelengths. The anisotropic case, on the other hand, is over-complicated and ignores the correlation among wavelengths.

A Problem Definition

Suppose that for a coal sample, there is a spectrum with D dimensions. The d-th dimension represents the reading for the d-th wavelength, where d=1; 2; . . . , D. If all D readings are put into a column vector x, x will be a D-dimensional input vector for the regression task. During training, N training samples {xₙ, yₙ}ₙ₌₁^{N} are given, each with a spectrum xₙ and the ground truth target value yₙ (e.g., H2O or heatan). The task of training is to learn a regression function f(x)=y.
During testing, the spectrum x is given and y is predicted to be y=f(x).

From Linear Ridge Regression to Kernel Ridge Regression

Linear ridge regression solves the following optimization problem

$${min}_{w} {\sum }_{n = 1}^{N} {( {w}^{T} {x}_{n} - {y}_{n} )}^{2} + λ {‖ w ‖}^{2} \qquad ( 1 )$$

w is a D-dimensional coefficient vector. The first term in (1) penalizes large regression errors. The second term is the regularization term to avoid overfitting. λ balances between error and regularization. It is easy to prove that the solution to (1) is

w=X^{T}(XX^{T}+λI)⁻¹Y (2)

where matrix X=[x₁; x₂; . . . ; x_{N}]^{T} and matrix Y=[y₁; y₂; . . . ; y_{N}]^{T}. For a test input x, its target value is estimated by

y=x^{T}w=x^{T}X^{T}(XX^{T}+ζI)⁻¹Y (3)

Kernel ridge regression extends from linear ridge regression by playing the kernel trick. Specifically, every inner product between two inputs encountered in (3) xₙ^{T}xₘ is now replaced by a Gaussian kernel k(xₙ, xₘ):

$$k ( {x}_{n} , {x}_{m} ) = \{ \begin{matrix}exp ( - γ {‖ {x}_{n} - {x}_{m} ‖}^{2} ) & isotropic case \\ exp ( - {\sum }_{d = 1}^{D} {{γ}_{d} ( {x}_{nd} - {x}_{md} )}^{2} ) & anisotropic case\end{matrix} \qquad ( 4 )$$

γ or γ_{d} is the kernel parameter. Using the kernel trick, (3) becomes

y=x^{T}w=k(x,)(K+λI)⁻¹Y (5)

where k(x,)=[k(x, x₁), . . . , k(x, x_{N})]. The kernel matrix K consists of Kₙₘ=k(xₙ,xₘ). It can be proved that the kernel ridge regression (KRR) as described in “[1] S. An, W. Liu, and S. Venkatesh. Fast cross-validation algorithms for least squares support vector machine and kernel ridge regression. Pattern Recognition, 40(8):2154-2162, 2007” is equivalent to a Gaussian process (GP) as described in “[2] C. E. Rasmussen and C. K. I. Williams. Gaussian Processes for Machine Learning. MIT Press, 2006.”

Parameterizing Kernel Parameters

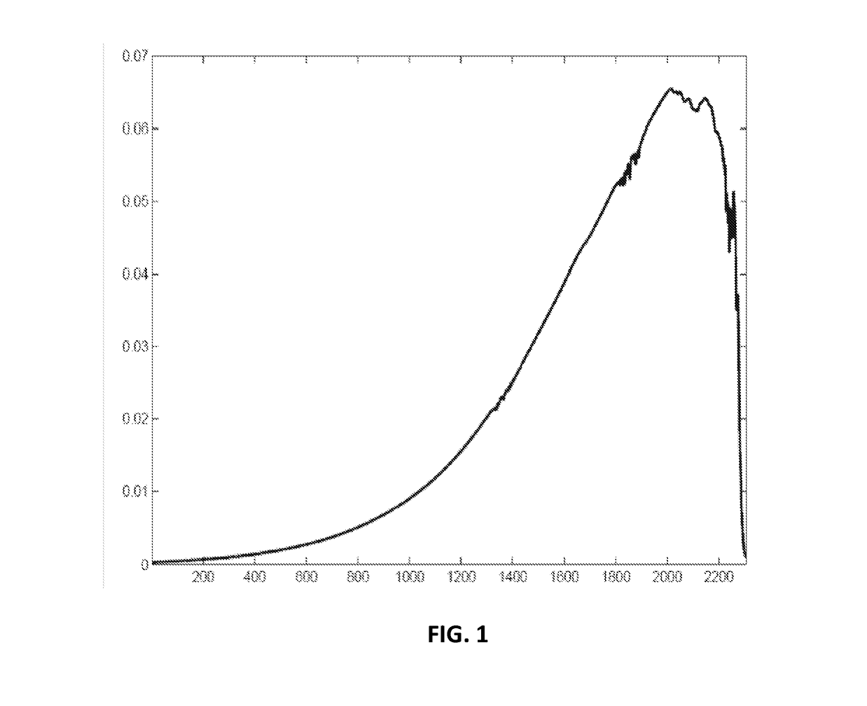

In the anisotropic kernel function (4), first a weighted squared distance between two inputs is calculated, with each dimension weighted by γ_{d}. Determining the weight γ_{d} is one step of the method. Consider the fact that adjacent spectrum values are different but correlated as shown in FIG. 1, which shows an example spectrum (x with dimension D=2307).

One may give similar kernel parameters γ_{d} to similar (neighboring) wavelengths. Neither using a single γ for all wavelengths (isotropic case) nor using an independent γ_{d} for every wavelength (anisotropic case) uses this fact well. Therefore, the anisotropic kernel function is extended by providing in accordance with an aspect of the present invention a new way to determine γ_{d} for the d-th wavelength (dimension).

The known wavelength information associated with each spectrum is used. Specifically, the wavelength for the d-dimension of the spectrum is provided by the spectroscopy as a function l(d), where d=1; 2; . . . ; D. For example, in a test dataset, the first wavelength l(1)=800.4 nm (nanometer) and the last wavelength l(2307)=2778.8 nm. In accordance with an aspect of the present invention it is required that γ is a smooth function over d. This smoothness can be enforced by a parametric form such as a polynomial function or a Gaussian function. But any smooth function that is positive over the applied domain will work. In accordance with an aspect of the present invention a smooth function is determined that provides favorable results.

Many parametric functions can be used here. One possible choice is a squared polynomial function

$$γ ( d ) = {( {\sum }_{k = 0}^{k = K} {α}_{k} {l}^{k} ( d ) )}^{2} )$$

where αₖ and K are the coefficient and degree of the polynomial function, respectively. The squared form in the above expression is to make sure that γ(d)≧0.

One option is to apply a Gaussian function. In accordance with an aspect of the present invention a Gaussian function is applied to define the smooth function for γ_{d}, which is determined by the following expression:

γ(d)=γ₀ exp(−β(l(d)−l₀)²) (6)

A Gaussian function emphasizes a certain range of wavelengths while dampening the rest, which appears to be a realistic choice. There are three extra parameters in (6). γ₀ represents the maximum value of γ(d) achieved at center l₀. β (similar to the role of γ_{d} in (4)) indicates the decay rate with regard to the squared distance of a wavelength from the center l₀.

Accordingly, a new anisotropic kernel function with γ_{d} in (4) replaced by the new smooth function γ(d) in (6) has been provided in accordance with an aspect of the present invention. Note that the isotropic kernel is a special case of the new kernel when β approaches zero and γ≈γ(d)≈γ₀.

Training Procedure

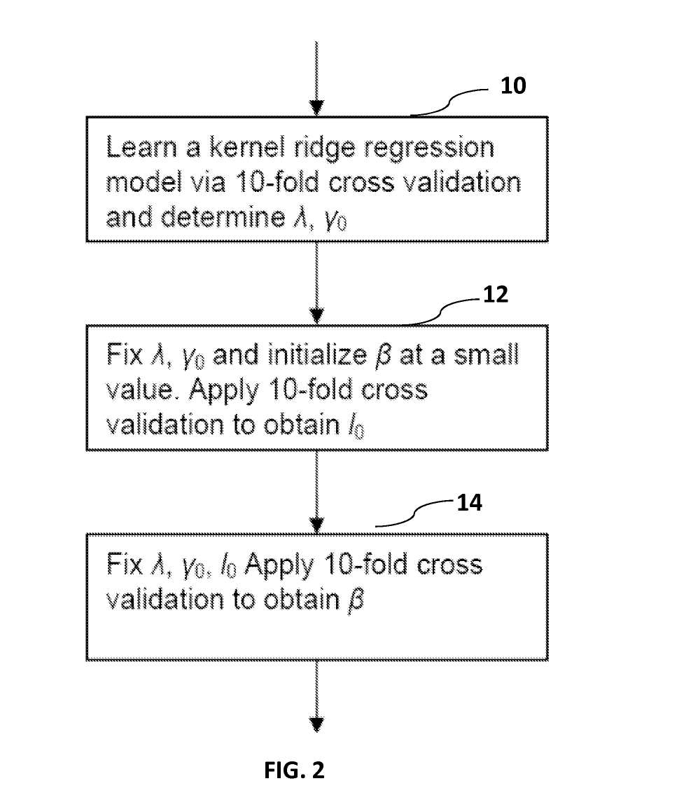

In accordance with an aspect of the present invention all four parameters (λ, γ₀, l₀ and β) are learned from training data. The method for this is initialized with the kernel ridge regression (KRR) under the isotropic case, which is trained using 10-fold cross validation. After the KRR is trained, λ in (3) and γ₀ in (6) are determined. See step 10. Next, β is fixed at a small value so the shape of γ(d) is relatively flat. Then the center location l₀ is varied and the best l₀ is picked via another 10-fold cross validation. See step 12. Finally, λ, γ₀, and l₀ are fixed and search for the best β is searched via a third 10-fold cross validation. See step 14. Alternatively, one can optimize all four parameters jointly using only one 10-fold cross validation. But this will be more time consuming FIG. 2 illustrates the work flow of the training procedure as described above.

Test Results

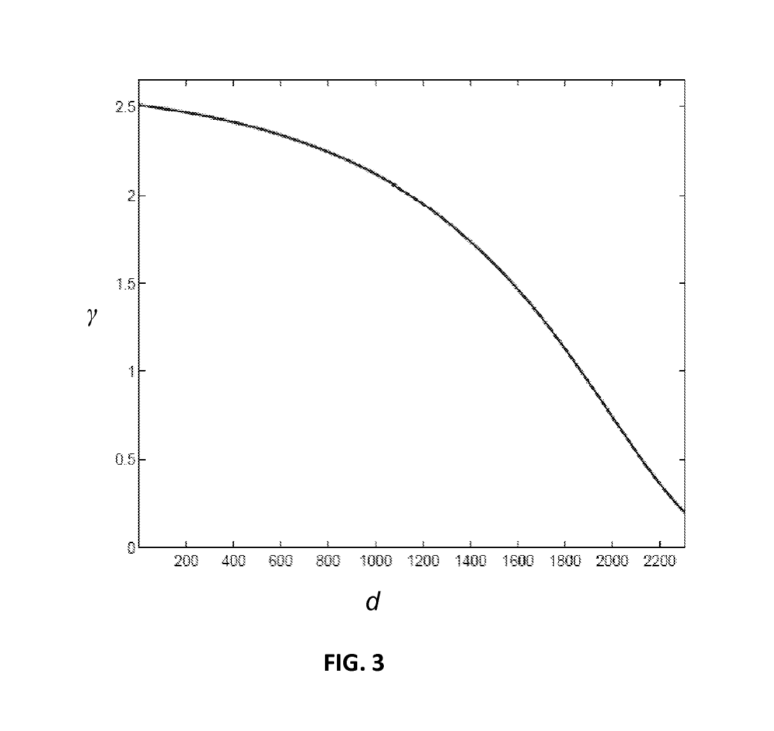

In one test a focus is on predicting heatan from a spectrum with D=2307 wavelengths ranging from 800.4 nm to 2778.8 nm. The training set consists of N=887 samples. After training, the parameters have the following values: λ=10⁻⁵, γ₀=2.626, l₀=500 and β=5.0×10⁻⁷. FIG. 3 illustrates γ(d) as a function of dimension index d. This result demonstrates that a smaller wavelength has a higher weight in the kernel function (4).

The above method is compared with KRR using 10-fold cross validation of the data. This process is randomly repeated 10 times. The root mean squared error (RMSE) is used for evaluation. There are a total of 10×10=100 errors. The average RMSEs (with standard deviation) for the new method and KRR are 1643.7(372.3) and 1742.2(698.9), respectively. The p value of a one-sided t test is 0.034, which indicates that the improvement of the new method over KRR is statistically significant.

Reconstructing Unknown Spectrum Wavelengths from Near-infrared Spectroscopy

Near-infrared (NIR) spectroscopy, being a relatively inexpensive, rapid, and non-destructive means of data collection is enabling many industrialists and academics the opportunity to increase the experimental complexity of their research, which in turn results in more accurate and precise information of their area of interest.

One of the possible fields of NIR spectroscopy usage is the coal industry (including coal mining, coal power, etc.). NIR spectroscopy is useful to overcome certain limitations, especially in a complicated real process, where on-line measuring is important to monitor the quality of coal. The NIR spectrometers satisfy the requirements of users who want to have quantitative product information in real-time because the NIR instrument provides the information promptly and easily. Multivariate statistical methods (linear and non-linear), which process enormous amounts of experimental data, have boosted the use of NIR instruments.

In real-world applications not all NIR instruments output spectra at exactly the same wavelengths due to the time, cost and convenience concerns. For example, compared to the NIR instruments which cover approximate 1200 nm to 2850 nm wavelengths, the instruments covering 1200 nm to 2250 nm wavelengths are much more inexpensive and easy-to-handle. This poses a machine learning issue: when training data has more features (i.e., spectrum wavelengths in one problem) than test data, how can the target values (i.e., calorific value in our problem) still be effectively predicted? Of course one can just select the features which appear in both training and testing to build a predictive model, but in this manner some valuable features of the training data may be lost. Furthermore, is it effective to use the additional training data? And is there any way to improve the accuracy of target prediction by integrating the unused features in the training data?

A novel approach in accordance with an aspect of the present invention is provided to reconstruct the features which appear in training data but not in test data. The features appearing in both training and testing are used to predict each of features only appearing in training data. Then the original features and the predicted features of the test data are combined to build a predictive model for the target. In this manner, the relationship is captured between the known and unknown features, thus paving the way for using the features which appear only in training data but not in test data. It is noted that the original features in the training data that do not appear in the test data thus do not overlap.

It is further noted that in one embodiment of the present invention the training data and the test data are obtained with the same or similar NIR spectroscopy devices, but in the testing phase fewer features are recorded than in the training phase. In another embodiment of the present invention, training data and test data are obtained with different NIR spectroscopy devices and the range of operation for obtaining the test data does not support or enable to obtain data in the range that is enabled by the NIR device for the training data.

Reconstruction Description

Assume each instance from the test data Xₜₑₛₜ is represented as a vector of feature values w₁, w₂, . . . , wₖ, i.e., Xₜₑₛₜ=(w₁, w₂, . . . , wₖ). Instead, each instance from the training data Xₜᵣₐᵢₙ is represented as a vector of feature values w₁, w₂, . . . , wₖ, wₖ₊₁, wₖ₊₂, . . . , wₖ₊ₜ, i.e., Xₜᵣₐᵢₙ=(w₁, w₂, . . . , wₖ, wₖ₊₁, wₖ₊₂, . . . , wₖ₊ₜ). Thus, wₖ₊₁, wₖ₊₂, . . . , wₖ₊ₜ are the features which appear in training data but not in test data.

One of known multivariate statistical methods is applied to reconstruct each feature wₖ₊ᵢ (where i=1, . . . , t) from the known features w₁, w₂, . . . , wₖ by modeling the relationship between feature sets {w₁, w₂, . . . , wₖ} and {wₖ₊₁, wₖ₊₂, . . . , wₖ₊ₜ} of training set. For training set, t regression models g₁, g₂, . . . , gₜ are built so that wₖ₊₁=g₁(w₁,w₂, . . . , wₖ), wₖ₊₂=g₂(w₁, w₂, . . . , wₖ), . . . , wₖ₊ₜ^{pred}=gₜ(w₁, w₂, . . . , wₖ). When given a new example x∈Xₜₑₛₜ, the predicted features are the outputs of these models, i.e., wₖ₊₁^{pred}=g₁(w₁,w₂, . . . , wₖ), wₖ₊₂^{pred}=g₂(w₁, w₂, . . . , wₖ), . . . , wₖ₊ₜ^{pred}=gₜ(w₁, w₂, . . . , wₖ). Next, the test data are updated by combining the known features and the reconstructed features, i.e., the updated test data as Xₜₑₛₜ′=(w₁,w₂, . . . , wₖ,wₖ₊₁^{pred},wₖ₊₂^{pred}, . . . , wₖ₊ₜ^{pred}).

In this manner, the updated test data have exactly the same features as the training data, we can apply the selected multivariate statistical methods to predict the target value. i.e., we build a regression model based on Xₜᵣₐᵢₙ and their target Yₜᵣₐᵢₙ where Yₜᵣₐᵢₙ=f(Xₜᵣₐᵢₙ). When given a new example x∈Xₜₑₛₜ, the predicted target value of this example is y_{pred}=f(x).

Note that in one test, both g and f are kernel ridge regression as described in “[1] S. An, W. Liu, and S. Venkatesh. Fast cross-validation algorithms for least squares support vector machine and kernel ridge regression. Pattern Recognition, 40(8):2154-2162, 2007.” It should be clear to one of ordinary skill that any multivariate statistical method can be applied for these models.

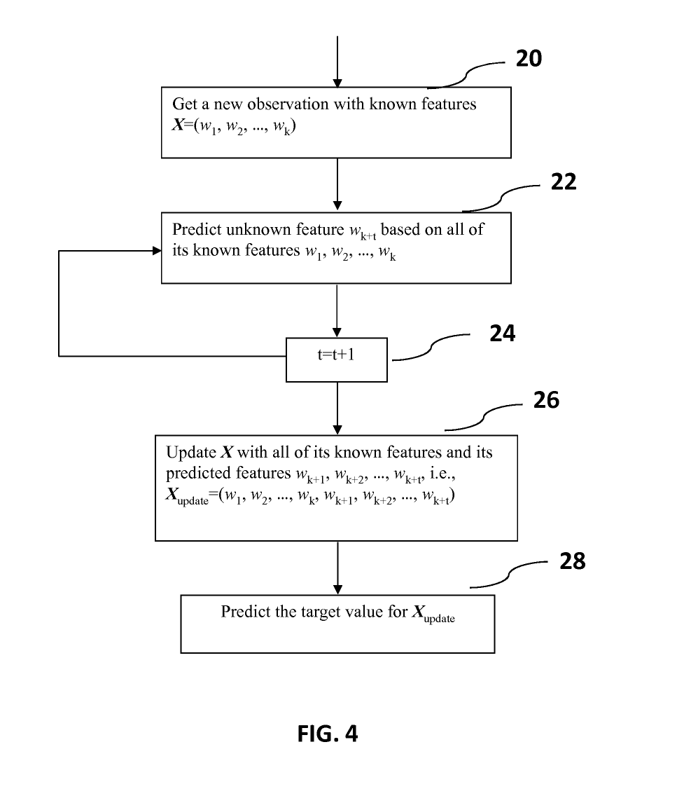

The feature reconstruction method performed by a processor is illustrated in FIG. 4. The new observation with known features is obtained in step 20. The unknown feature is predicted in step 22. As indicated in step 24, the step 22 is repeated a number of times. In step 26, the X is updated with its known features and its predicted features. In step 28, the target value for X_{update} is predicted.

Test Results

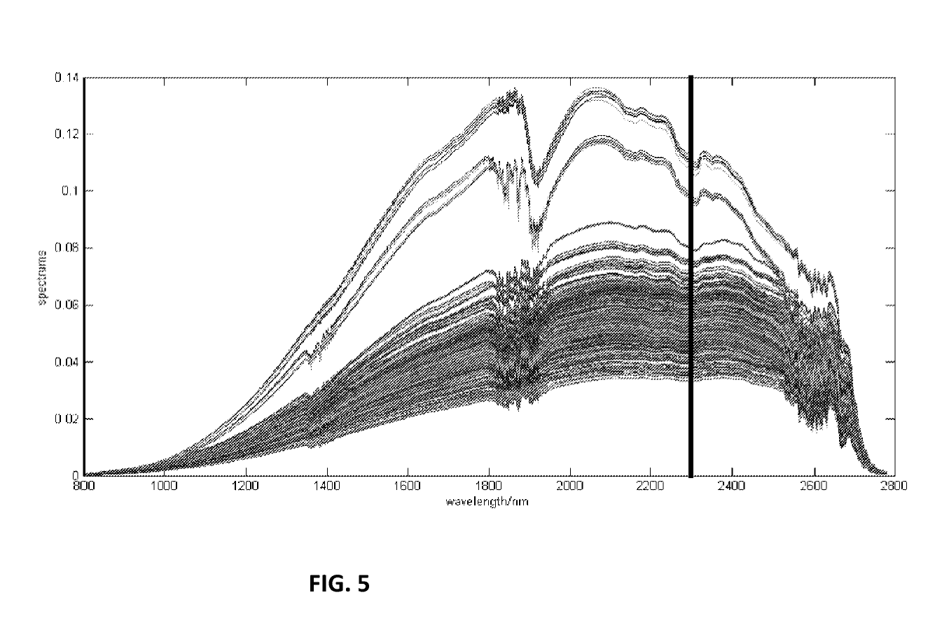

In an illustrative example the method provided herein in accordance with an aspect of the present invention is demonstrated using real-world NIR data of coal. The data contains 887 samples and 2307 features. These 2307 features correspond to 2307 waves with wavelength ranging from 800 nm to 2800 nm These 887 samples belong to 221 coals (i.e., each coal contains 4-5 samples). The goal is to predict the calorific value of each coal sample based on NIR spectrums. FIG. 5 shows the spectrum information of 887 samples.

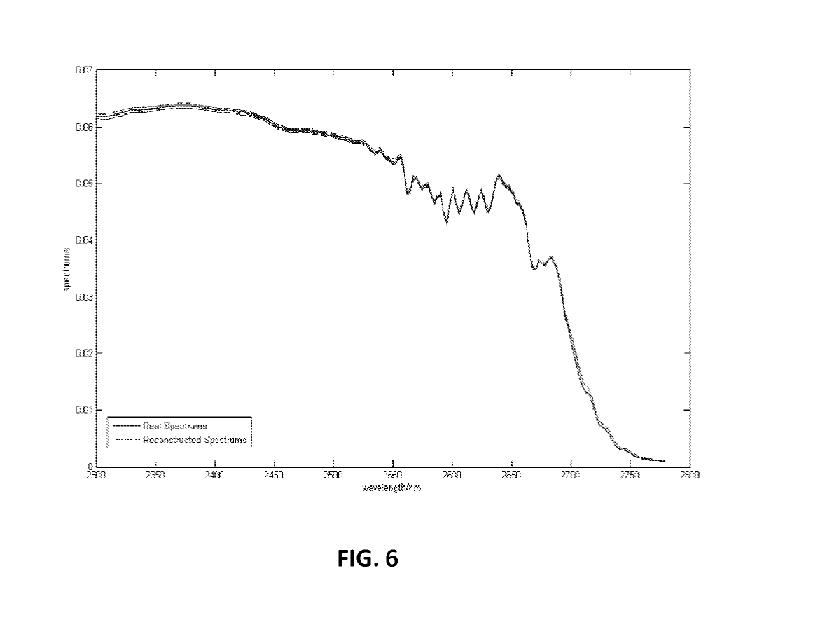

A practical circumstance is simulated: the full length waves are not available. For example, only the waves with wavelength range from 800 nm to 2300 nm are obtained (2112 features, the left side of the vertical line in FIG. 5). By using the reconstruction method provided in accordance with one or more aspects of the present invention, the unknown waves features ranging from 2300 nm to 2800 nm (195 features) are reconstructed. The statistical method used for reconstruction is kernel ridge regression. The feature reconstruction results for coal ‘MPA KL01 Herne Aug Vic Ballast 110303 befeuchtet’ is plotted in FIG. 6 which clearly shows the property of the herein provided method in accordance with one or more aspects of the present invention: the real spectrums are well depicted by the reconstructed ones as the reconstructed and the actual spectrum almost completely coincide.

To test the effectiveness of the reconstruction method on the prediction of calorific value, the known features (here are the waves with wavelength shorter than 2300 nm) and the reconstructed features (here are the predicted waves with wavelength between 2300 nm and 2800 nm) are combined for all samples from the test data. Then also kernel ridge regression was applied to predict the calorific value for each sample from the test data. A leave-one-out strategy was used to evaluate the performance of the herein provided reconstruction method. Root Mean Square Error (RMSE) was applied to measure the prediction accuracy.

The RMSE is calculated as RMSE=\sqrt{Σₗ₌₁^{N}(yₗ−yₗ)²/N}, where y is the predicted value, y is the true value, and N is the total number of samples. When only the 2112 features were used from the waves with wavelength 800 nm to 2300 nm, the RMSE is 1751±1569; when both the 2112 features and the 195 reconstructed features which were predicted from the 2112 known features were used, the RMSE is 1609±1094, i.e., an 8.8% improvement in accuracy was obtained.

To further characterize a property of the reconstruction method, the calorific value prediction results of kernel ridge regression were compared with and without our newly proposed reconstruction process for different wavelength thresholds. For example, wavelength<2300 means that only the waves with wavelength shorter than 2300 are used to build the predictive model. Table 1 summarizes the results when the chosen thresholds are 2100, 2200, 2300, 2400, 2500, and 2600. Table 1 clearly shows the advantage of the herein provided reconstruction methods: without resort to the unknown features, the herein provided method improves the calorific value prediction at all tested situations.

TABLE 1
Comparison of calorific value prediction without and
with feature reconstruction

| | W/O Feature | With Feature |
|---|---|---|
| Different Wavelengths | Reconstruction | Reconstruction |
| Wavelength < 2100 nm | 2008 ± 1484 | 1952 ± 1607 |
| Wavelength < 2200 nm | 1910 ± 1489 | 1783 ± 1339 |
| Wavelength < 2300 nm | 1751 ± 1569 | 1609 ± 1094 |
| Wavelength < 2400 nm | 1739 ± 1540 | 1700 ± 1311 |
| Wavelength < 2500 nm | 1718 ± 1386 | 1672 ± 1286 |
| Wavelength < 2600 nm | 1779 ± 1524 | 1686 ± 1451 |

The results show that reconstructing unknown spectrum Wavelengths successfully boosts the coal quality prediction, which is very useful when the available spectrum wavelengths are very limited. An innovative approach to reconstruct the features which appear in training data but not in test data has been provided herein in accordance with an aspect of the present invention. The proposed approach models the features appearing in both training and testing to predict each of features only appearing in training data, then combines the original features and the predicted features of the test data to build a predictive model for the targets. The herein provided method can be used in conjunction with any multivariate statistical method in real-world applications.

The method was tested on a NIR data of coal for predicting calorific values. The results show that the method successfully captures the relationship between the known and unknown NIR spectrums and improves the prediction accuracy by 8.8% compared to the procedures without the feature construction approach. It is believed that this is the first successful approach to reconstruct unknown spectrum wavelengths from NIR data. The provided approach saves money and time while improving coal quality prediction when applied to real-world NIR data.

Improving Regression Quality on Near-Infrared Spectra Data by Removing Outliers

It is difficult to directly measure the contents of coal, such as H₂0 and heatan. One popular method is to build a multivariate regression model using the infrared spectral properties of the coal. The chemical and physical properties measured by Near-Infrared (NIR) spectroscopy are regarded as the independent variables. These independent variables are denoted as X. The contents or properties of the coal are regarded as dependent variables. Currently, these dependent variables are studied separately. Denote y as one type of dependent variables. One goal is to build a high quality regression model f(x) mapping X to y based on the training set as was explained earlier above. Then the resulting regression model f(x) can be used to predict the coal contents for new samples with the same type of NIR measurements.

Outlier Removal and Prediction

In practical situations, outliers are often contained in NIR spectra data, which may be caused by the instrument, operation or sample preparation. These outliers would degrade the quality of the regression model significantly. There are two types of outliers in an analysis: (1) input space outliers (noise is introduced to independent variables X); (2) output space outliers (noise is introduced to the dependent variable y). One focus herein in accordance with an aspect of the present invention is on removing output space outliers from training set. Experimental results show that the technique of outlier removal provided herein in accordance with an aspect of the present invention improves the accuracy of predicting heatan values of coals by 10% compared to the baseline method without outlier removal. The herein provided technique is simple but effective. It can be easily applied to any regression algorithm.

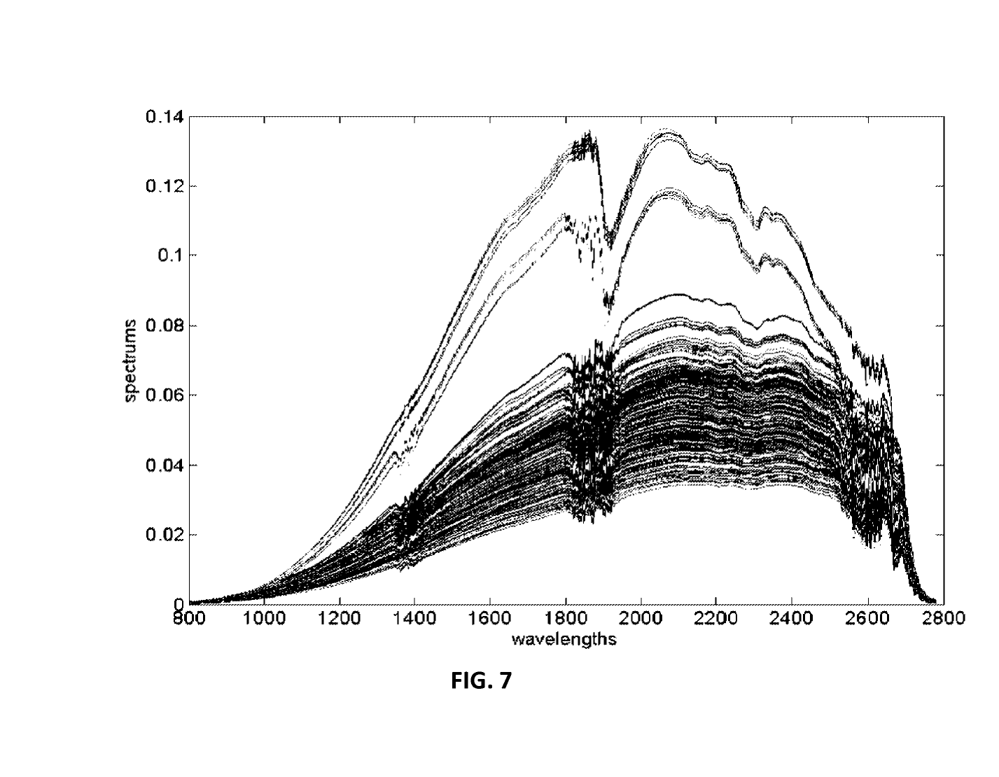

Denote xᵢ={xᵢ₁, xᵢ₂, . . . , x_{id}} as the NIR spectra measurements of the i-th example, where d denotes d different wavelengths. One example of NIR data for coal is given in FIG. 1. In this specific example, the number of wavelengths is 2307. These wavelengths range from 800 nm to 2800 nm FIG. 7 shows the spectrums of 887 samples. For each sample xᵢ, a target value yᵢ is associated with it. Given a training dataset D={(xᵢ, yᵢ), i=1, . . . , N}, one goal is to build a regression model y=f(x). Then, with any new test example x, its target value can be predicted as ŷ=f(x). Many robust regression algorithms, such as Principal Component Regression (PCR), Partial Least Square regression (PLS) as described in “[5] S. Wold, H. Rube, H. Wold, and W. J. Dunn III. The collinearity problem in linear regression. the partial least squares (pls) approach to generalized inverse. SIAM Journal of Scientific and Statistical Computations, 5:735-743, 1984” and Kernel-based PLS regression (KPLS) as described in “[3] Roman Rosipal and Leonard J. Trejo. Kernel partial least squares regression in reproducing kernel hilbert space. Journal of Machine Learning Research, 2:97-123, 2001” are widely used in NIR data. However, these approaches mainly focus on removing the noise contained on independent variables.

In a regression problem of NIR data, the noise is also introduced to the dependent variable y. With the noise introduced on dependent variable y, the function f(x) learned based on the training data set D can not be generalized well to the test set.

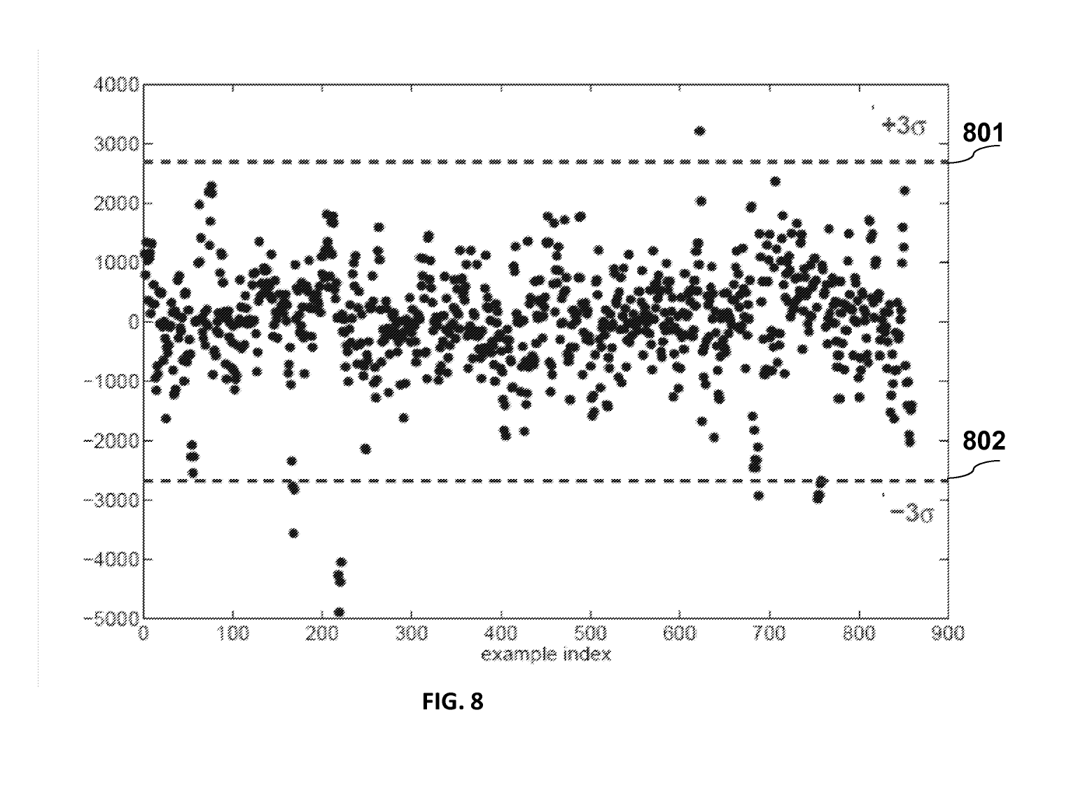

In accordance with an aspect of the present invention the output space outliers are removed from the training set using a 3σ edit rule: if the training error of the i-th example is out of the range of ±3σ, it will be regarded as an outlier and it is removed from the training set from which the regression model is built. FIG. 8 shows a plot of training errors. Two stepwise lines 801 and 802 in FIG. 8 indicate the boundary of ±3σ. As shown in this figure, the training examples with training error out of the ±3σ boundary will be treated as outliers. These outliers will be removed from the training set. This means that not only the target value but also the relates NIR sample data will be removed, so that a new regression model that is calculated does not depend on the removed data.

The training error of the i-th example is calculated as

errᵢ=yᵢ−ŷᵢ,

where ŷᵢ=f(xᵢ) is the predicted value of the i-th example, γᵢ is the true value of the i-th example. Given the training errors {err₁, err₂, . . . , errᵢ, . . . , err_{N}}, the standard deviate a can be computed as

$$σ = \sqrt{\frac{{\sum }_{i = 1}^{N} {( {err}_{i} - \overline{err} )}^{2}}{N}} ,$$

where

$$\overline{err} = \frac{{\sum }_{i = 1}^{N} {err}_{i}}{N}$$

is the average of the training errors. A normal distribution of the training errors is assumed.

According to the 3σ edit rule:

Pr( e̅r̅r̅−3σ≦err≦ e̅r̅r̅+3σ)≈0.9973,

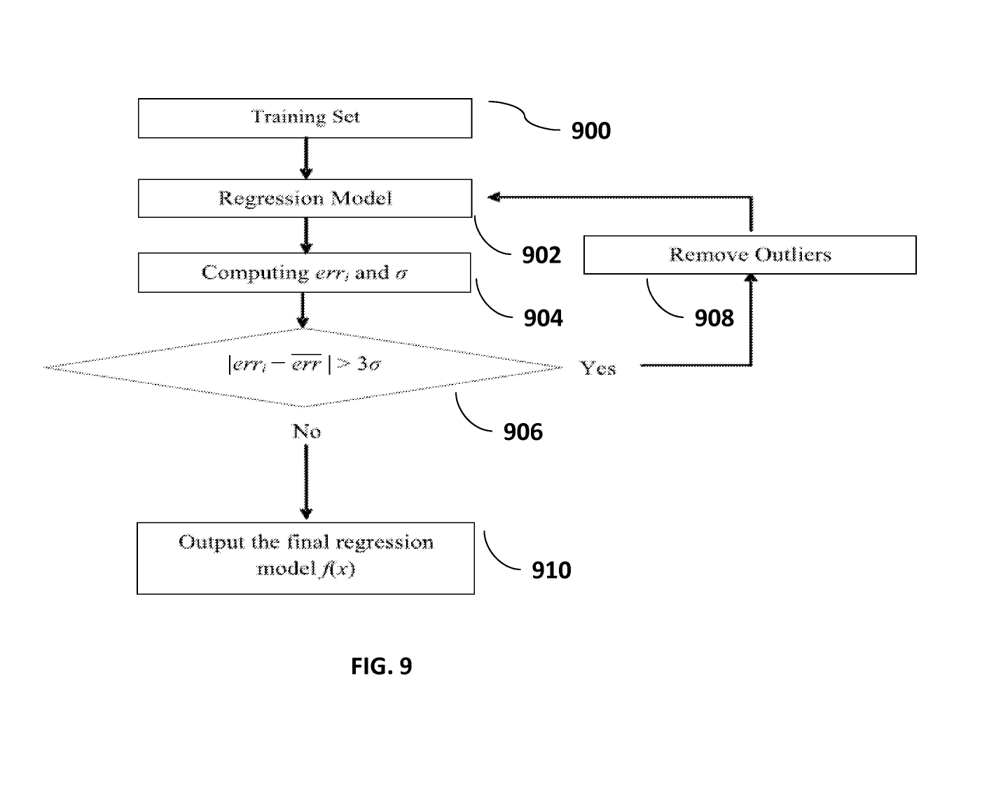

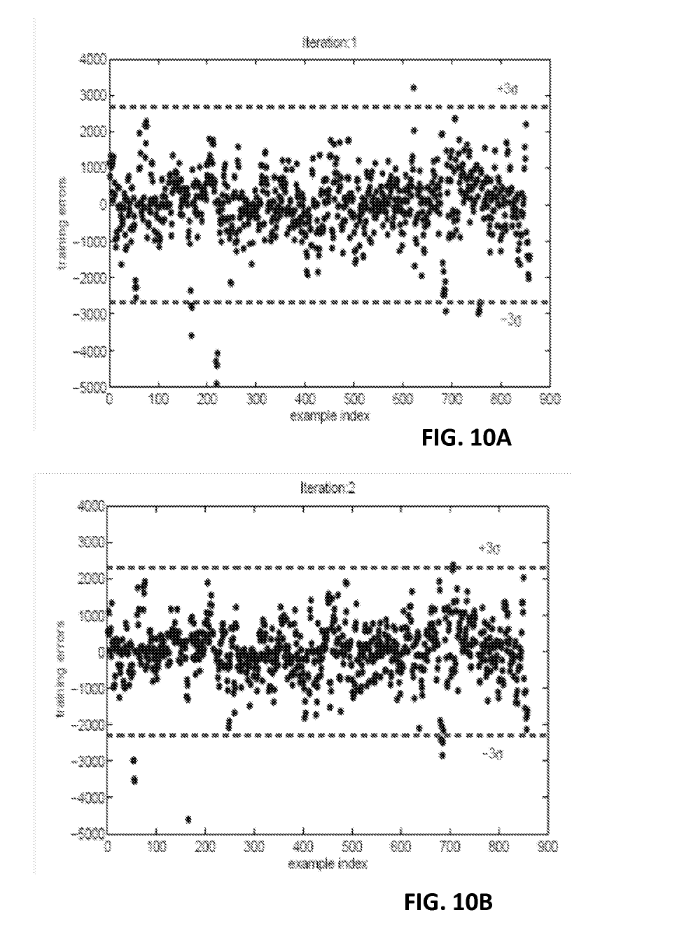

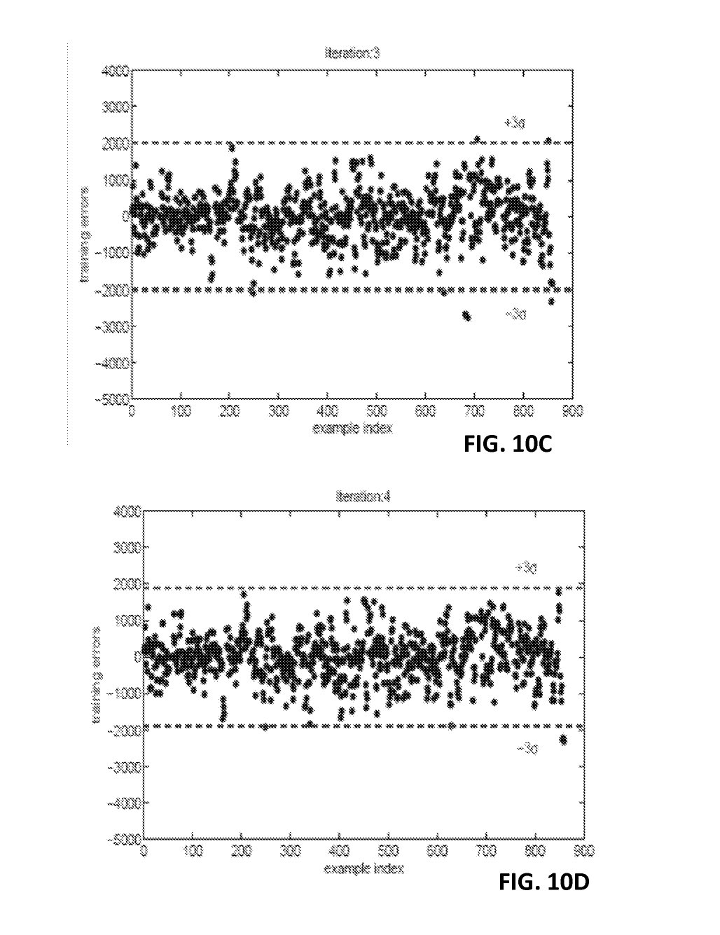

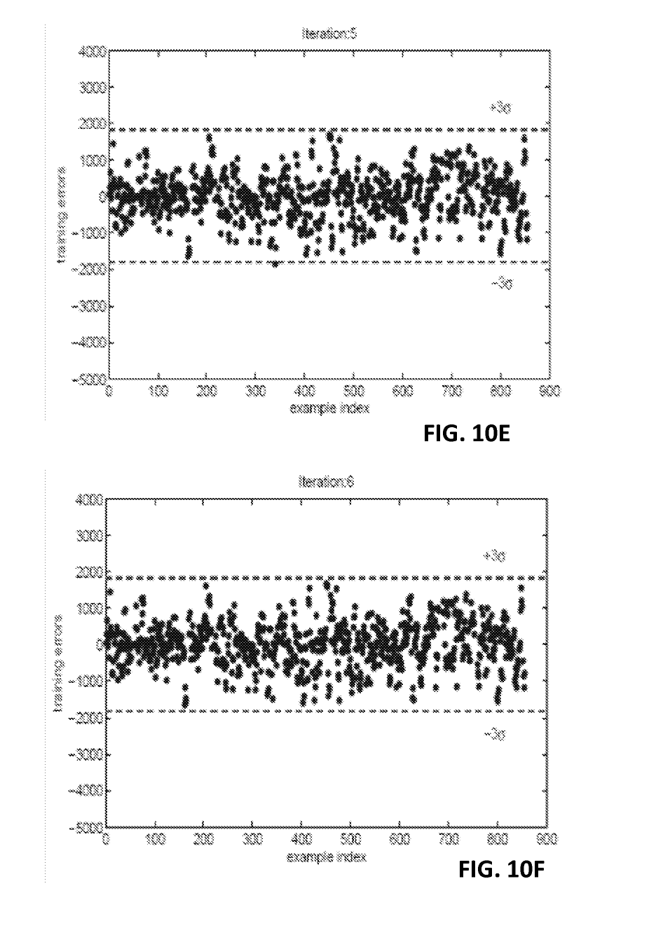

e̅r̅r̅±3σ reflects a significant level at 0.003 to detect a training example as an outlier. Therefore, the i-th example is regarded as an outlier and removed from the training data set if |errᵢ− e̅r̅r̅|≧3σ. Since the removal of outliers reduces the standard deviation of training errors, the 3σ edit rule is applied in iterative manner until all training errors are within the ±3σ region. The framework of the outlier removal method is illustrated in FIG. 9. FIGS. 10A-10F illustrate the iterative steps of removing outliers from the training set. In FIGS. 10A-10F, the outliers are found above and below the dotted lines. The calculation continues until all the outliers are removed, as shown in FIG. 10F. The process of removing as illustrated in the diagram of FIG. 9 is called pruning of the training data.

Kernel Ridge Regression

A brief overview of the Kernel Ridge Regression Algorithm will be provided. Kernel ridge regression is used in an analysis because: (1) It can capture the non-linearity of the data; (2) There exist formulas to compute the leave-one-out Root Mean Square Error (RMSE) using the results of a single training on the whole training data set. Therefore, the hyper-parameters can be optimized efficiently; (3) It obtained the best empirical results based on a preliminary analysis.

Given a training data set D={(xᵢ,yᵢ), i=1, . . . , N}, the N×N kernel matrix K can be calculated as Kᵢⱼ=κ(xᵢ,xⱼ), where κ(,) denotes a positive semi-definite (psd) kernel function. By using the representer theorem as described in “[4] B. Scholkopf, R. Herbrich, and A. J. Smola. A generalized representer theorem. In Proceedings of the 14th Annual Conference on Computational Learning Theory, pages 416-426, 2001,” the regression function is spanned by training data points.

Therefore, the prediction values of the training examples can be expressed as f=Kα, where α with size N×1 represent the kernel expansion coefficients. The optimization objective of kernel ridge regression is given by

min∥Kα−y∥²+λαKα.

Here, y denotes the true target value of the training examples. The λ is a regularization parameter. The close form solution of kernel ridge regression is

α=(K+λI)⁻¹y.

Therefore, the prediction value of an unseen test example x is given by

f(x)=K(x,)α

where K(x,) denotes the Kernel similarity between the test example x to all training examples {xᵢ}ᵢ₌₁^{N}.

Test Results

The performance of the method provided herein in accordance with an aspect of the present invention is tested on a real-life NIR dataset of coal. This coal dataset contains 887 samples and 2307 features. These 2307 features correspond to 2307 waves with wavelength ranging from 800 nm to 2800 nm. These 887 samples belong to 221 coals. So, each coal has 4-5 samples. One goal is to predict the coal contents, such as H₂O and heatan, based on the NIR measurements. The samples that belong to the same coal have slightly different spectrums but the same target value. Therefore, the samples are split into training and test set based on coals.

The leave-one-out cross validation (LOOCV) strategy is used to evaluate the performance of the proposed algorithm. So, at each fold, one coal is used as test set and the rest are used as training set. The RMSE is used to measure the prediction accuracy. The RMSE is calculated as:

$$R M S E = \sqrt{\frac{{\sum }_{i ∈ S} {( {y}_{i} - {\hat{y}}_{i} )}^{2}}{\vert S \vert}} ,$$

where S denotes the test set and |S| is the size of the test set.

The method herein provided with an aspect of the present invention was compared with a baseline KRR algorithm. The baseline KRR algorithm would not perform well because outliers are contained in the coal dataset. The Gaussian Kernel was applied in the experimental setting herein. The kernel similarity between xᵢ and xⱼ is computed as K(xᵢ, xⱼ)=exp(−∥ᵢ−xⱼ∥²*γ). The two hyper-parameters λ and γ in KRR are chosen as follows: λ∈{10⁻⁷,10⁻⁶, . . . , 10³}, γ∈γ₀*2^{{-4,-3, . . . , 4}}, where γ₀ is the reciprocal of the averaged distance between each data points to the data center. The optimal value for λ and γ are chosen based on leave-one-out cross validation on training set.

The procedures of iteratively removing outliers in the training set in accordance with an aspect of the present invention is illustrated in FIG. 9. First, the training set is obtained in step 900. A regression model is developed from this set in step 902. The deviation and error are calculated in step 904. In step 906, it is determined if there are outliers based on a threshold value. In step 908, if outliers are detected, they are removed in step 908, creating a reduced training set which is used to create a new regression model in accordance with step 902. When no outliers are detected, the process stops in step 910. As indicated in FIG. 9, the standard deviation σ is decreased when outliers are removed from the training set. A reduced training set is obtained so that all training errors are within a threshold region such as a ±3σ region. Then, a regression model is built on the reduced training set. The LOOCV experimental results for predicting two different target values (i.e., H₂O and heatan) are shown in the following Table 2.

TABLE 2

| | H₂O | Heatan |
|---|---|---|
| Baseline (KRR) | 0.244 ± 0.218 | 1,220 ± 1,119 |
| Herein provided method | 0.244 ± 0.228 | 1,099 ± 1,116 |

As shown in Table 2, the herein provided method improves the accuracy of predicting heatan by 10%. The performance of KRR and the proposed method on predicting h2o is similar.

Based on the feedbacks from domain experts, the RMSE on predicting h2o is good and acceptable. This supports the assumption that the outliers are mainly caused by noise introduced to dependant variable y. So, significant improvement is achieved on prediction on heatan but not on H₂O.

Dimension Reduction

As shown in FIG. 7, the wavelength variables are highly correlated. So, it is desirable to further improve the regression performance on predicting heatan by applying PCA to preprocess the NIR data. The new experimental results are presented in Table 3.

TABLE 3

| | Original data | nComp = 50 | nComp = 100 | nComp = 150 |
|---|---|---|---|---|
| Baseline (KRR) | 1,220 ± 1,119 | 1,242 ± 1,139 | 1,222 ± 1,102 | 1,223 ± 1,087 |
| Novel method | 1,099 ± 1,116 | 1,091 ± 1,150 | 1,076 ± 1,137 | 1,091 ± 1,125 |

As shown in Table 3, the herein provided method is always better than the baseline KRR. Another interesting observation is that selecting different number of principle components would not affect the regression performance too much.

The herein provided method of iteratively removing outliers from a training data set in accordance with another aspect of the present invention is combined with the also herein provided method of smoothing the kernel parameters. Accordingly, first a regression model kernel is created from training data using the smoothing function. Next, the smoothed kernel based model is applied to training data to determine and remove the outliers as explained above.

The herein provided method of iteratively removing outliers from a training data set in accordance with another aspect of the present invention is combined with the also herein provided method of reconstructing wavelength dependent features. In accordance with an aspect of the present invention first the features are reconstructed as explained herein and next

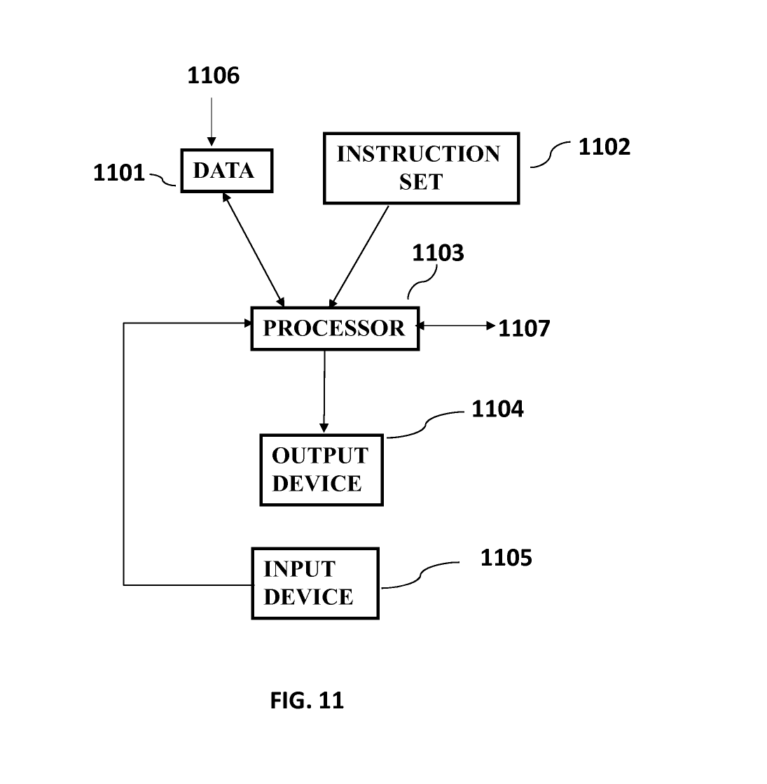

The methods as provided herein are, in one embodiment of the present invention, implemented on a system or a computer device. Thus, steps described herein are implemented on a processor in a system, as shown in FIG. 11. A system illustrated in FIG. 11 and as provided herein is enabled for receiving, processing and generating data. The system is provided with data that can be stored on a memory 1101. Data may be obtained from an input device. Data may be provided on an input 1106. Such data may be spectroscopy data or any other data that is helpful in a quality measurement system. The processor is also provided or programmed with an instruction set or program executing the methods of the present invention that is stored on a memory 1102 and is provided to the processor 1103, which executes the instructions of 1102 to process the data from 1101. Data, such as spectroscopy data or any other data provided by the processor can be outputted on an output device 1104, which may be a display to display images or data or a data storage device. The processor also has a communication channel 1107 to receive external data from a communication device and to transmit data to an external device. The system in one embodiment of the present invention has an input device 1105, which may include a keyboard, a mouse, a pointing device, or any other device that can generate data to be provided to processor 1103.

The processor can be dedicated or application specific hardware or circuitry. However, the processor can also be a general CPU or any other computing device that can execute the instructions of 1102. Accordingly, the system as illustrated in FIG. 11 provides a system for processing data and is enabled to execute the steps of the methods as provided herein in accordance with one or more aspects of the present invention.

While there have been shown, described and pointed out fundamental novel features of the invention as applied to preferred embodiments thereof, it will be understood that various omissions and substitutions and changes in the form and details of the methods and systems illustrated and in its operation may be made by those skilled in the art without departing from the spirit of the invention. It is the intention, therefore, to be limited only as indicated by the claims.

1. A method for determining a property of a material from data generated by a near-infrared spectroscopy device, comprising:
obtaining wavelength based training data related to the material;
a processor using the wavelength based training data to learn an anisotropic Gaussian kernel function with a wavelength based kernel parameter that is defined by a smooth function over the wavelength determined by at least one parameter; and
the processor applying the anisotropic Gaussian kernel function to wavelength based test data of one or more samples of the material generated by the near-infrared spectroscopy device to determine the property.

2. The method of claim 1, wherein the smooth function is a smooth Gaussian function and the at least one parameter is a decay parameter.

3. The method of claim 1, wherein the material is coal.

4. The method of claim 1, wherein the property is heatan.

5. The method of claim 2, wherein the wavelength based kernel parameter that is defined by a smooth Gaussian function over the wavelength, is expressed as:
γ(d)=γ₀ exp(−β(l(d)−l₀)²), wherein:
d is an index value related to the wavelength;
γ(d) is the wavelength based parameter;
γ₀ is a maximum value of the wavelength based parameter;
β is the decay parameter;
l(d) is the wavelength at index value d; and
l₀ a wavelength value for which the wavelength based parameter reaches the maximum value.

6. The method of claim 5, further comprising:
the processor learning a kernel ridge regression for an isotropic kernel from the training data;
the processor determining a regularization factor and γ₀;
the processor applying an initialization value for β and determining l₀; and
the processor determining an operational value for β.

7. The method of claim 6, further comprising:
the processor applying the kernel ridge regression to the wavelength based training data to determine a first plurality of target values;
the processor determining a standard deviation from the first plurality of target values;
the processor identifying a reduced plurality of sets of training data by removing at least one set of training data from the wavelength based training data based on the standard deviation; and
the processor applying the kernel ridge regression to the reduced plurality of sets of training data to determine a second plurality of target values.

8. A method to reconstruct a feature in test data related to a material obtained with a near-infrared spectroscopy device, comprising:
storing on a memory near-infrared spectroscopy training data from the material including data of a first and a second set of features which do not overlap;
creating with a processor a predictive feature model to predict features appearing in the second set of features in the training data from the first set of features in the training data by using the first and second set of features in the training data;
obtaining with the near infra-red spectroscopy device test data from the material including test data related to the first set of features; and
predicting a second set of features related to the test data of the material by applying the predictive feature model.

9. The method of claim 8, further comprising:
combining the first set of features and the predicted second set of features related to the test data to create a predictive model for a property of the material.

10. The method of claim 8, wherein each first set of features relates to a first range of wavelengths in NIR spectroscopy and each second set of features relates to a second range of wavelengths in NIR spectroscopy.

11. The method of claim 8, wherein the first range of wavelengths includes wavelengths shorter than 2300 nm and the second range of wavelengths includes wavelengths greater than 2300 nm.

12. The method of claim 8, wherein the predictive feature model is based on a multivariate statistical method.

13. The method of claim 12, wherein the multivariate statistical method is a kernel ridge regression method.

14. The method of claim 9, wherein the material is coal and the property is a calorific value.

15. A method for determining a property of a material with data generated by a spectroscopy device, comprising:
a processor receiving a first plurality of sets of training data generated by the spectroscopy device;
the processor generating a regression model from the first plurality of sets of training data to determine a first plurality of target values, which is representative of the property of the material;
the processor determining a standard deviation from the first plurality of target values;
the processor identifying a second plurality of sets of training data by removing at least one set of training data from the first plurality of sets of training data based on the standard deviation; and
the processor generating a regression model from the second plurality of sets of training data to determine a second plurality of target values.

16. The method of claim 15, further comprising:
the processor generating a regression model from a remaining plurality of sets of training data to determine a remaining plurality of target values;
the processor determining a new standard deviation from the remaining plurality of target values; and
the processor determining if any of the sets of training data of the remaining plurality of sets of training data should be removed based on the new standard deviation.

17. The method of claim 16, wherein none of the sets of training data is removed from the remaining plurality of sets of training data and the regression model based on the remaining plurality of sets of training data is applied by the processor to determine a target value from a set of test data generated by the spectroscopy device.

18. The method of claim 15, wherein the material is coal and the spectroscopy device is a near-infrared spectroscopy device.

19. The method of claim 15, wherein the removing of at least one set of training data from the first plurality of sets of training data is based on a 3σ range.

20. The method of claim 15, wherein the property is a calorific value of coal.
